SEQUENCE LISTING (3 sequences)

SEQ ID NO: 1 - DNA, 8351 bp, Homo sapiens
  source 1..8351: mol_type = genomic DNA
ccatcctctttcccgctcctcgctctcggacgccaccaacgctctcccgggctcttccgcgttacctacgatggaaggtcggggcgtgcgggcagctggaacccacccctgtcttggagctccgggtagctctcaaactcgaggctgcgcaccccctttcccgtcagctgagctctagagcgctggggctttctttttgatcgacttcttgaaataaaaccaactttatcattctttgggtaacagacccaaaagccgatgggacggcccgctgggctgttcccgcccctatgcccttttttgggtttccggccagaggcatgctgggagcgtcacatgcaaattgagcgtgcacccagcgttccgccctttctacgctgggccggttatcgacccggcccagtgcgcaggcgcgggaaagttgaactaataaagtttgtacgagttcagtggaggagaccgcaagttgagtggaggaggcggcggtggggccccggaccaggtcagcggggtgttgacgaggggtggggtgaggagggaagaggagggggccgggatccagtggtgggcaccccagtcccgcttctctgatatccaagggtccagggtcctctagattccagcaggggtcggagatttggaactacacctcacagcaggctgcgcggcgaagaagcgcggacctaggggaagatttgccctgaaacctcttgggattggtagttcccgatgctttaagggtttcaggagtttggcatatgcctcttatagaatccaggagttggggcgctaagcctcttcttcctgggagacctaagggtctggggctccagttcctttttctctaagactcagaagtccaggaccccagctccctcctcctttagacccacgaatctgggcacccagccccaccacctttccttggatcgaagagtctttttcctgatcgctttttcgtctgaggactcaagagtctaggcccccagacccacactctccaaaggacccaggagtccaggccctcagcaccttccttgggactcacaagtcctggtccccaagcctccacttcttgctaaacccttggagtccagtccctaaccctctctctcacaggtgcctccatggcaggctctgaagagctggggctccgggaagacacgctgagggtcctagctgccttccttaggcgtggtgaggctgccgggtctcctgttccaactccacctaggtaagaggagtggcccttctcccccaggggccagcatttgaggctccctcctaactcttgctcctggtctcagtctgtctcctctctttatatctggctctattctctgtctttggggttttggagagaggtcctcagcgggggagggagggagttggggaaagagactgcttgcaggggatctctggtggggtcagtctctgaagtcctcctctccagaagccctgcccaagaagagccaacagacttcctgagccgccttcgaagatgtcttccctgctccctggggcgaggagcagccccctctgagtcccctcggccttgctctctgcccatccgcccctgctatggtttagagcctggtaagagatttccatgatcatctatgaagccggcagaacacgggataaggtgctagtttaagatcactcagctagggggtggctgtgtggagtctctagcaccagccttcaggacagaaccgtcctgtcctctcccctccccagttagatttctgtcaattctgtgggttggctttcagagagcatttggtcaatctggttgttgtctctttccttccataactacttccctgcccctaggacttttgtagccaggaatacccctgatagaggggcagaagggctggttttcaatcacatctccctggctgttttagttgcgtgactctggggaaaatgctttctcttccctgctcccctgtttttccaatagcagagtaggaattggggaggaggtcactggacttgatcaaggtgacccacaagtttcctcataggtgtgaacctggacagactagtaatggctgctgggagtgctagcttgaaaaggggttcacagcttgggagaggaagtgctgtgatcaattggtgatgtctgtagtgagtgagggaaggggatactaacagtaatgtcttgtgtttgccttccttggatgagatcatcttcaaggtctagttgagctctgacagtcttttggagacagattgttgctctgtcacccaggctggagtgcaggggtgcaatctcgactcactgcaatccccacctcctgggttcaagcgattctcgtgcctcagcctcccgagtgactgggattacaggtgcacgccagcatactagctgatttttatacttttagtagacacggggtttcgccacgttggccaggctggcctcaaactcctggcctcaagtgaccctcccacgtcggcctcccaaaatgttgagattgcagacgtgagccaccgcaccctgtcagctctgacattcttttttttttttttttttttgagacggagtttcgcccttgttgcccaggctggagtgcaatggcccgatctcggctcaccacaacctctacctcccgggttcaagtaattctcctgcctcagcctcccaagtagctgggattataggcatgcgccaccatgcccggctaattttttttgtatttttagtagagatggggtttctccatgttggtcaggctgctctcgaactcccaacctcaggtgatctgcctgcctcagcctcccaaagtgatgggattacaggcgtgagccactgcgcctggccccagctctgacattctaagtccagtctgaggatctcataaagtgaaaagtacaagggttttggcatttttaaccaagttttctcactgggcctcagtttaccccattataaaatgggaatcatggccaggcttggttgctcacgcctgtaatcctagcactttgggaggctgaggtgggtggattgcctgaggtcagcctgggcaacacggtgaaaccccatttctactaaaatacaaaaaattagccaggtatggcggcgtgcgcctgtagtcccagctactcatgaggctgaggcaggagaattgcgagactccgtttcccaaaatcaatacataaataaataataaataaaatgggaatcattgccacctgccaaggtagttgggggaaggagagaatccgtaagtttccaatgtcctggaaacctgtcattaactcttttcaccccaggcccagctactccagacttctatgctttggtggcccagcggctggaacagctggtccaagagcagctgaaatctccgcccagcccaggtgaggcacagaggagccccagagaaggatggcggtgggaggatagtcagggttctgcccccagccaaattctcttctgcctccagaattacagggtcccccatcgacagagaaggaagccatactgcggaggctggtggccctgctggaggaggaggcagaagtcattaaccagaaggtgatgggcatctgtcccactccttggcaaggacaggagttgcggaatggtggggcactgggatcagaagctggatctgtattttctttggatggtcaggtggaaagaggctgggacaaggtttcaatgaagggttgaggccgggtgtggtggctcacgcctgtaatcccagcactttgggaggccgagttgggtggatgacttgaggtcaggagttcgagaccagcctggccaacatggtgaaaccccatctctactaaaaatacaaaaatcggacaggtgtggtggtgcgcttctgtaatcccagctactcagaaggctgaggcaggagaatcgcttgagcccaggaggtggaggttgcagtgagccaagattgcgccactgcactccagcctgggtgacagagtgagactctgtctttaaaaaaaaaaaaaaaaaagaatagttgaatgacaggggtatggttagggtagtactgtaccgtaaattagttgttttaattgattgtttagaggggcatggcctaaatgcagagggtgtggccatcagaaacattatccacaagagactggccaagctggggaagtaagtgaatgaatgtgggggtggggggagtggctaaaatcttactgagcatagaagggacctggcttagaatgtaggggaaaatgtactaccagaggaggtgtggcttgtggctgtgtcctggaatctgattggcttcggagcttggacaaggtacatcgtcctttaatattaattgaatggtagagggcaagattgttgtagacactaggttggcccctgggaaaggtcaccaggggcatggctagattaactcagttacagtagagattggccctgattggctgttgaggcaagggcctggcatggcttagggactcacgttccaggccactatcccgggggtagtttggcagagaccctggagtctggcttgattggctaatatggatgaggggtggcctagaaccttagcatctagaacgatcctgaaccgggaagccacgcctcccggccctctattggctggccccgggggcgctgctgacgcggaccctgcctcttccaccctacagctggcctcggaccccgccctgcgcagcaagctggtccgcctgtcctccgactctttcgcccgcctggtggagctgttctgtagccgggatgacagctctcgcccaagccgagcatgccccgggcccccgcctccttccccggagcccctggcccgcctggccctagccatggagctgagccggcgcgtggccgggctggggggcaccctggccggactcagcgtggagcacgtgcacagcttcacgccctggatccaggcccacgggggctgggtgagccgctgaagcctctctctccgggcctcactttacctaactgtcatgtgatagaggaggggcggggacttcttaggttcctctcagacctcaggtattctcccagctccactgcgttcgttctgttgagcccttgctgtatgccaggactgcagcgatgagtgcagacactatgcctggcgtcaacgactgggtacgctaatactgtgagcgtgaaaccagtgactgaggttactcagggagaggccggacggggtagctcatgcctgtaatcccagcacttttggaggccaaggcaggagaattatttgaggccaggagttcaagaccagcctggacaacatagtaagaccccatctctacaaaaattgaaaataactagccagggtcgggtgcggtggctcacacctgtaatcccagcactttgggaggccgaggcaggtggatcacctgaggtcaggagtttgagaccagctggccaacgtggtgaaaccctgtgtctactaaaaaaaatacataaattagccgggcgcggtggcaggtgcctgtaatcccagctaccgaggagactgaggcatgagaatcgcttgaacctgggaggcagagattgcagtgagctgagatcatgccactgcactccagcctgggcgacagagtgagactgtgtctcaacaaaataatataataaaaacctggtgcggtggctcacacctgtaatcccagcactttgggagactgaggtgggtggatcacttgaggtcaggagtttgagaccagcctgaccatggtgacattcagtctctactaaaaatacaaaattagcccggcatgcctgtaatcccagctagggaggctgaggcaagagaattgcttgaacctgggaggcagaggttgtagtgagccgagattgcaccactgcactccagcctgggcaacaagagcggggctctgtctcaaaaaataaataaataaaaatttttaaaattaataataattagcaaagcatggtagctcacacgtttactcccagctactcaggaggcagaggagccgggaggatcacttgagcccaggagttcaaggctgtggtgagctatgattgcgccactgtactccagtgtgggtgacagagtgggaccctgtctcaaaaaataaaatagtgagagtagtagtgctacctcaaagggtcgctctgagggttaaaatggggtcaatgtggtaagccctctgtgcactgcctggcatgcaggcattttgttggtgaggttatatggctacctgctgcctttccaacagtgcctgccaggatgccctcccggggaaccccctgcctccgtcctgctccttctggtgggctctggtttcacccccaacgaggccttcccaggtccccattgacaatcacagccacctgcacaccctcccaccgccctccttcccccttcctgctttacttttccttccatagcactttgggccttccaacagatggtggaattgacttgttttacttgtcttctggtttatttttctttgttggtgtctcccacctctaacacatcagcttcaggagacccagaagtcttcccctctccttttcttactttatctccagtgccggtttcagagttggattgggctccagcgtgctggccctgtggcttggcgactgtcgcagtctccctgagcggcactctccccactggtccagtgagcaccgtctcatttacctgccttggggggtcgctgtgggtattcactgagagagggacacaagccaggctctggatggaaccggtttccctggcaggagctggtcaagtgtgtttgaggagcagggaggaggaggccagtagggctacagtagggtgagcgggcgacagatgagaggaggagaggcgccgggccagccaggaccctgtggtcacggtgagtgacaactcggactttgactctgggtaagccaaggtctctgagcagcggagggacaggcgctgatttaggcgctaatgggcgccctctggcggctgcctggggacagactgctgggggcgagggcagaagtaggggcacagggaggaggtgagtggatctcacccaagtcgggggcggtggagaatgctggatttaacttagcggtgaagctgacagatttggctaatgggccagatgtggggacagactaaaggatgctggcccgagcagctgcagggcgacatcttaagttccccgagatggggaaggtgtggggatgggggcgatgggggtggggagcaggactggaggtggtccggtctacagtaagatcagaactcatagcacatgttgaggggaatggatataagtgttgggctcagcaaagaagctcgtttattttattttattttaatttttcagatggagtctcactctattgcccagactggagtgcagtggcacgaccttggctcactgcaacctccacctcctgggttcaagcgattccctgcctcagcctcctgagtagctggggttacaggcgcacaccaccatgtccggctaattttgtagttttagcagagacggggttttgccctgttggccaggctggtctcgaactcttggcctcaagtgatccacccacctcggcctcccaaagtgctggaattacaggctgagctaccacgcctggacaatttcttaaaaaatttttttaaagtaggattgatagcagatgagttttctcggtgggccaggtcctctgctagggatttcaatggaatgatcccatttaaacccactacaacctcatggggctcttattgtcctcgtcttgtgggggcagagaggaaaagtccccagcatgcagagtgttggaatctggatttgtacacaaagccacgactctgaattgttcaaatcccaagactctcttccccatctctgactctgtcccccaactcttcactctctctggatctctgtctaatcccactccagtgtcctgaccctcctcgagctcaacctttcctcctcctgctaggaccctgccccctcatccctgtgcccttctgtctgggtctctgtccccttgtctgggtgtctgtcaccctccgctctctggttcctcgccttcccgtctctaatctcacagggctgatgtggacgctgctgtatggggggaacacctgccctgctcacagggctctgccacttttcccttccaggagggcatcctggctgtttcacccgtggacttgaacttgccattggactgagctctttctcagaagctgctacaagatgacacctcatgtccctgccctcttcgtgtgcttttccaagtcttcctattccactcagggctgtggggtggtggttgccctacctgtttttgccaaaaataaattgtttaaaacttttcttattaaaaacgttacaaagta

SEQ ID NO: 2 - DNA, 24 bp, synthetic construct
  misc_feature 1..24: note = antisense oligonucleotide
  source 1..24: mol_type = other DNA
gaaatctcttaccaggctctaaac

SEQ ID NO: 3 - DNA, 22 bp, synthetic construct
  misc_feature 1..22: note = antisense oligonucleotide
  source 1..22: mol_type = other DNA
catagatgatcatggaaatctc

(19) United States
(12) Patent Application Publication
(10) Pub. No.: US 2025/0019707 A1
(43) Pub. Date: Jan. 16, 2025

(54) ANTISENSE OLIGONUCLEOTIDE FOR REGULATING BCL2L12 EXPRESSION AND THE USE THEREOF IN TREATMENT OF DISEASES

(71) Applicant: SHANDONG UNIVERSITY, Shandong (CN)

(72) Inventors: Zhaojian LIU, Shandong (CN); Zixiang WANG, Shandong (CN); Junchao QING, Shandong (CN); Ling ZHAO, Shandong (CN); Xiyu ZHANG, Shandong (CN)

(21) Appl. No.: 18/715,856

(22) PCT Filed: Dec. 2, 2022
(86) PCT No.: PCT/CN2022/136357

§ 371 Date: Aug. 12, 2024

(30) Foreign Application Priority Data
Dec. 3, 2021 (CN) 202111464202.8

(51) Int. Cl. C12N 15/113; A61P 35/00

(52) U.S. Cl. CPC C12N 15/1135; A61P 35/00; C12N 2310/11; C12N 2310/315; C12N 2320/33

(57) ABSTRACT

An antisense oligonucleotide for regulating BCL2L12 expression, and a method and drug for treating BCL2L12-related diseases.

This application claims the priority of Chinese patent application CN 202111464202.8 submitted on Dec. 3, 2021, with the title of the invention is “Antisense oligonucleotide for regulating the expression of BCL2L12 and its use in treating diseases”, the entire content of which Incorporated in this application by reference.

### TECHNICAL FIELD

The invention relates to the fields of biotechnology and disease treatment. More specifically, the present invention relates to antisense oligonucleotides for regulating the expression of BCL2L12, as well as methods and medicines for treating BCL2L12-associated diseases.

### TECHNICAL BACKGROUND

Splicing is a highly dynamic process. The spatiotemporal assembly process of the spliceosome and the interaction with precursor RNA and the interaction between RNA polymerase II and the spliceosome in the physiological state of higher organisms need to be further studied. Compared with lower organisms, in human cells, the number of splicing factors involved in RNA splicing has increased significantly, the number of pre-mRNA and splicing events has also greatly increased, and the regulatory process is more refined. In addition, in human cells, the coupling of gene transcription and splicing has also enhanced the complexity of splicing (Montes, Sanford et al.2019). How to accurately screen and identify disease-specific splicing isoforms in disease states is also a huge challenge for future research. Studies have shown that the number of splicing events in tumors is 30% more than that in normal tissues (Kahles, Lehmann et al.2018), and abnormal splicing events in tumors are involved in the malignant behavior of tumor cells, promoting tumor cell proliferation, invasion and metastasis, anti-apoptotic ability, recurrence and drug resistance (Inoue, Chew et al. 2019, Zhou, Wang et al. 2019). It has been reported in the literature that CD44 (Tjhay, Motohara et al. 2015) and FBXW7 (Xu, Zhuang et al. 2020) genes have abnormal splicing in ovarian cancer and are associated with prognosis.

BCL2L12 (BCL2-Like 12) belongs to the BCL2 family and is a proline-rich protein that contains a BH2 domain and is involved in the regulation of apoptosis. Due to alternative splicing, BCL2L12 produces a long splicing isoform, BCL2L12-L, and a short isoform, BCL2L12-S.

Alternative splicing can be used as a drug target for the treatment of rare diseases, tumors and other diseases, and antisense oligonucleotides (ASO) are powerful tools for regulating alternative splicing. In recent years, antisense oligonucleotides have broad application prospects in the field of drug research and development because of their good specificity and rapid action, and their mechanism of action and administration methods are also being continuously reported. Fu Xiangdong et al. found that the use of antisense oligonucleotides that inhibit PTB in astrocytes can directly transdifferentiate astrocytes into functional neurons, which provides a promising future for treatment strategy for neurodegenerative diseases (Qian, Kang et al. 2020). The world's first drug for the treatment of spinal muscular atrophy was approved by the FDA in 2016. In addition, Spinraza is an antisense oligonucleotide that can change the splicing of the SMN2 gene, increase the expression of fully functional SMN protein, and significantly improve the motor function of patients with spinal muscular atrophy (Jafar-Nejad, Powers et al. 2021).

There are currently no effective drugs targeting alternative splicing for BCL2L12. There is a need in this field to research and develop methods and drugs for controlling the occurrence and development of related diseases by regulating the alternative splicing of BCL2L12.

### SUMMARY OF THE INVENTION

The inventors of the present invention have discovered that the key apoptosis-related gene BCL2L12 undergoes abnormally splicing in tumors (including ovarian cancer, etc.), and the core transcript of BCL2L12 differs in exon 3, which produces a long transcript (BCL2L12-L) and a short transcript (BCL2L12-S) due to splicing, and discovered a canonical GU splice site around the junction area of exon 3 and intron 3. The inventor thus designed and verified an effective sequence capable of regulating the splicing mode of BCL2L12, thereby providing a method and a drug capable of inducing tumor cell apoptosis, inhibiting cell proliferation and treating tumors.

Specifically, the present invention provides method of modulating a function of and/or the expression of BCL2L12 polynucleotides in mammalian cells or tissues in vivo or in vitro, comprising: contacting said mammalian cells or tissues with an antisense oligonucleotide 5 to 40 nucleotides in length, wherein said antisense oligonucleotide has at least 50% sequence identity to a reverse complement of a natural sense of a BCL2L12 polynucleotide, thereby modulating a function of and/or the expression of the BCL2L12 polynucleotides. In the present invention, the BCL2L12 polynucleotide can be the genomic DNA of BCL2L12, especially refers to the single strand (sense strand, or coding strand) of the nucleotide sequence carrying the information of the encoded protein in the genomic DNA. The antisense oligonucleotide provided by the present invention is reverse complementary to the sequence of mRNA (including mature or immature precursor-mRNA or pre-mRNA) transcribed from genomic DNA, and can recognize and bind to the mRNA (including pre-mRNA), and thereby regulate (interfere with) the normal functions of nucleic acids, such as splicing and translation.

In one aspect of the present invention, the length of the antisense oligonucleotide is about 5-40 nucleotides, such as about 5 to about 30 nucleotides, about 10 to about 30 nucleotides, About 15 to about 25 nucleotides or about 20-25 nucleotides, such as 22 nucleotides, 23 nucleotides or nucleotides.

BCL2L12, that is, BCL2-like protein 12, the protein encoded by it belongs to a member of the protein family containing Bcl-2 homology domain 2 (BH2) (Stegh, Brennan et al.2010), encoded by the BCL2L12 gene (HGNC:13787). Due to alternative splicing of exon 3 of BCL2L12, BCL2L12 produces a long transcript (BCL2L12-L) and a short transcript (BCL2L12-S).

In one of the embodiments of the present invention, said antisense oligonucleotide has at least 50% sequence identity to a reverse complement of a polynucleotide comprising 5 to 40 consecutive nucleotides within a polynucleotide of SEQ ID NO: 1. The nucleotide sequence of SEQ ID NO: 1 is the sequence of positive strand of BCL2L12 genomic DNA, that is, the sequence of the single-stranded DNA identical to the transcribed mRNA sequence.

In one of the embodiments of the present invention, the antisense oligonucleotide has at least 50% sequence identity to a reverse complement of a natural sense of a polynucleotide fragment at the junction of exon 3 and intron 3 of the BCL2L12 polynucleotide. In the polynucleotide as shown in SEQ ID NO: 1, exon 3 is at positions 1454-1596, and intron 3 is at positions 1597-3285. In one of the embodiments of the present invention, the aforementioned antisense oligonucleotide has at least 50% sequence identity to a reverse complement of a polynucleotide comprising 5 to 40 consecutive nucleotides within the nucleotides 1567 to 1626 of a BCL2L12 polynucleotide of SEQ ID NO: 1.

In one of the embodiments of the present invention, the antisense oligonucleotide has at least 50% sequence identity to a reverse complement of a natural sense of the following polynucleotide fragments at the junction of exon 3 and intron 3 of the BCL2L12 polynucleotide: a fragment of about 5-40 nucleotides at the end of intron 3 connecting with exon 3, a fragment of about 5-40 nucleotides at the end of exon 3 connecting with intron 3, or a fragment of about 5-40 nucleotides at the junction of exon 3 and intron 3. In one embodiment of the present invention, said fragment of about 5-40 nucleotides has about 5 to about 30 nucleotides, about 10 to about 30 nucleotides, about 15 to about 25 cores nucleotides or about 20-25 nucleotides, such as 22 nucleotides, 23 nucleotides or nucleotides.

In one of the embodiments of the present invention, the antisense oligonucleotide includes (is) a reverse complement of a natural sense of the polynucleotide fragment of about 5-20 nucleotides at the end of intron 3 connecting with exon 3 of the BCL2L12 polynucleotide. Preferably, said antisense oligonucleotide includes (is) a reverse complement of a natural sense of the polynucleotide fragment of about 5 nucleotides at the end of intron 3 connecting with exon 3 of the BCL2L12 polynucleotide.

In one of the embodiments of the present invention, the antisense oligonucleotide has at least 50% sequence identity to a reverse complement of a polynucleotide comprising 5 to 40 consecutive nucleotides within the nucleotides 1597 to 1616 of a BCL2L12 polynucleotide of SEQ ID NO: 1, more preferably, said antisense oligonucleotide includes a reverse complement of the nucleotides 1597 to 1601 of a BCL2L12 polynucleotide of SEQ ID NO: 1.

Introns are portions of eukaryotic DNA which intervene between the coding portions, or “exons,” of that DNA. Introns and exons are transcribed into RNA termed “primary transcript, precursor to mRNA” (or “pre-mRNA”). Introns must be removed from the pre-mRNA so that the native protein encoded by the exons can be produced (the term “native protein” as used herein refers to naturally occurring, wild type, or functional protein). The removal of introns from pre-mRNA and subsequent joining of the exons is carried out in the splicing process. The splicing process is actually a series of reactions, mediated by splicing factors, which is carried out on RNA after transcription but before translation. Thus, a “pre-mRNA” is an RNA which contains both exons and intron(s), and an “mRNA” is an RNA in which the intron(s) have been removed and the exons joined together sequentially so that the protein may be translated therefrom by the ribosomes.

Introns generally includes one or more “splice elements” which are relatively short, conserved RNA segments which bind the various splicing factors which carry out the splicing reactions. Thus, each intron is defined by a 5′ splice site, a 3′ splice site, and a branch point situated therebetween. These splice elements are “blocked”, as discussed herein, when an antisense oligonucleotide either fully or partially overlaps the element, or binds to the pre-mRNA at a position sufficiently close to the element to disrupt the binding and function of the splicing factors which would ordinarily mediate the particular splicing reaction which occurs at that element.

In one of the embodiments of the present invention, the inventors have discovered that the key apoptosis-related gene BCL2L12 undergoes abnormally splicing in tumors (including ovarian cancer, etc.), and the core transcript of BCL2L12 differs in exon 3, which produces a long transcript (BCL2L12-L) and a short transcript (BCL2L12-S) due to splicing, and discovered a canonical GU splice site around the junction of exon 3 and intron 3. The inventors thus designed antisense oligonucleotides that recognize and bind to the junction fragments around the junction of exon 3 and intron 3 of BCL2L12 which prevent splicing factors to act and immature expression of downstream target genes through complementary base pairing. The inventors provides a stable and effective sequence capable of regulating the splicing mode of BCL2L12, thereby providing antisense oligonucleotides, methods and drugs that can induce tumor cell apoptosis, inhibit cell proliferation, and treat tumors.

In one of the embodiments of the present invention, said antisense oligonucleotide comprises one or more modifications selected from: at least one modified sugar moiety, at least one modified internucleoside linkage, at least one modified nucleotide, and combinations thereof. In one embodiment of the present invention, one or more nucleotides in the aforementioned antisense oligonucleotides are modified nucleotides. In one embodiment of the present invention, the one or more modifications comprise at least one modified internucleoside linkage selected from: a phosphorothioate, 2′-Omethoxyethyl (MOE), 2′-fluoro, alkylphosphonate, phosphorodithioate, alkylphosphonothioate, phosphoramidate, carbamate, carbonate, phosphate triester, acetamidate, carboxymethyl ester, and combinations thereof. The use of modified nucleotides can render the antisense oligonucleotides of the invention to have higher target binding affinity and/or nuclease resistance.

In one of the embodiments of the present invention, said antisense oligonucleotide has a nucleotide sequence as shown in SEQ ID NO:2 or SEQ ID NO:3.

In one of the embodiments of the present invention, said antisense oligonucleotide is:

G*A*A*A*T*C*T*C*T*TACCAGG*C*T*C*T*A*A*A*C;
or
C*A*T*A*G*A*T*GATCATG*G*A*A*A*T*C*T*C

(* stands for phosphorothioate modification).

In one of the embodiments of the present invention, the aforementioned antisense oligonucleotides are combined with the natural sense sequence of the BCL2L12 polynucleotide in the cells or tissues of the mammal and modulating on the BCL2L12-L transcript and the BCL2L12-S transcript. In one embodiment of the present invention, the aforementioned antisense oligonucleotide reduces the expression of BCL2L12 in the cells or tissues of the mammal.

The expression of BCL2L12 can be detected by any method known in the art for detecting protein (expression) in a sample. The method can check for the presence or absence of the protein. The method can also quantitatively detect the amount of expression of the protein.

Methods for detecting protein (expression) in a sample that can be used in the present invention include immunoassay. For example, ELISA or Western blotting with antibodies that specifically recognize the protein can be used. Antibodies can be monoclonal or polyclonal.

The method for detecting protein (expression) in a sample that can be used in the present invention also includes detecting the presence or amount of mRNA of the protein, for example, detecting the amount of mRNA or fragments of the protein in the sample by RT-PCR. RT-PCR methods and conditions for detecting mRNA of proteins are known or readily available to those skilled in the art.

All genes, gene names, and gene products disclosed herein are intended to correspond to homologs from any species for which the compositions and methods disclosed herein are applicable. Thus, the terms include, but are not limited to genes and gene products from humans and mice. In an embodiment, the genes or nucleic acid sequences are human.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “oligonucleotide” refers to an oligomer or polymer of ribonucleic acid (RNA) or deoxyribonucleic acid (DNA) or mimetics thereof. The term “oligonucleotide”, also includes linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Oligonucleotides are capable of specifically binding to a target polynucleotide by Watson-Crick type of base pairing.

By “antisense oligonucleotides” is meant an RNA or DNA molecule that binds to a “target nucleic acid”. the term “target nucleic acid” encompasses DNA, RA (comprising premRNA and mRNA) transcribed from such DNA, and also cDNA derived from such RNA, coding, noncoding sequences, sense or antisense polynucleotides. The specific hybridization of an oligomeric compound with its target nucleic acid interferes with the normal function of the nucleic acid. This modulation of function of a target nucleic acid by compounds, which specifically hybridize to it, is generally referred to as “antisense”. The functions of DNA to be interfered include, for example, replication and transcription. The functions of RNA to be interfered, include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of an encoded product or oligonucleotides.

In some embodiments, the sequence identity between the antisense oligonucleotide and the target is from about 50% to about 60%. In some embodiments, the sequence identity is from about 60% to about 70%, about 70% to about 80%, or about 80% to about 90%. In some embodiments, the sequence identity is about 90%, about 92%, about 94%, about 95%, about 96%, about 97%, about 98%, about 99%, or about 100%.

Target segments can include DNA or RNA sequences that comprise at least the 5 consecutive nucleotides from the 5 ‘-terminus of one of the illustrative preferred target segments (the remaining nucleotides being a consecutive stretch of the same DNA or RNA beginning immediately upstream of the 5’-terminus of the target segment and continuing until the DNA or RNA contains about 5 to about 100 nucleotides). Once one or more target regions, segments or sites have been identified, antisense compounds are chosen which are sufficiently complementary to the target, i.e., hybridize sufficiently well and with sufficient specificity, to give the desired effect.

The term “nucleotide” covers naturally occurring nucleotides as well as nonnaturally occurring nucleotides. Thus, “nucleotides” includes not only the known purine and pyrimidine heterocycles-containing molecules, but also heterocyclic analogues and tautomers thereof. Illustrative examples of other types of nucleotides are molecules containing adenine, guanine, thymine, cytosine, uracil, purine, xanthine, diaminopurine, 8-oxo-N6-methyladenine, 7-deazaxanthine, 7-deazaguanine, N4, N4-ethanocytosin, N6, N6-ethano-2,6-diaminopurine, 5-methylcytosine, 5-(C3-C6)-alkynylcytosine, 5-fluorouracil, 5-bromouracil, pseudoisocytosine, 2-hydroxy-5-methyl-4-triazolopyridin, isocytosine, isoguanin and inosine.

An antisense oligonucleotide is “specifically hybridizable” when binding of the compound to the target nucleic acid interferes with the normal function of the target nucleic acid to cause a modulation of function and/or activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

It is understood in the art that the sequence of an oligomeric compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure, mismatch or hairpin structure). The oligomeric compounds of the present invention comprise at least about 70%, or at least about 75%, or at least about 80%, or at least about 85%, or at least about 90%, or at least about 95%, or at least about 99% sequence complementarity to a target region within the target nucleic acid sequence to which they are targeted.

In the present invention, the mammal may be any kind of mammals, including and not limited to Rodentia (such as mice and rats), Lagomorpha (rabbits), Carnivora (felines and canines), Artiodactyla Order (bovines and suids), Persiscus (equines), or primates and apes (humans or monkeys). The mammal is preferably a human.

The present invention also provides a method for treating or preventing a BCL2L12-associated disease in a mammal, comprising: administrating to the mammal with an antisense oligonucleotide 5 to 30 nucleotides in length, wherein said antisense oligonucleotide has at least 50% sequence identity to a reverse complement of a natural sense of a BCL2L12 polynucleotide, thereby modulating a function of and/or the expression of the BCL2L12 polynucleotides. In one of the embodiments of the present invention, said antisense oligonucleotide has at least 50% sequence identity to a reverse complement of a polynucleotide comprising 5 to 40 consecutive nucleotides within a polynucleotide of SEQ ID NO: 1. In one of the embodiments of the present invention, said antisense oligonucleotide is as defined as aforementioned.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting it development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

In one of the embodiments of the present invention, said BCL2L12-associated disease is a cancer. Cancers include but not limited to: fibrosarcoma, mucinous sarcoma, liposarcoma, chondrosarcoma, osteogenic sarcoma, chordoma, hemangiosarcoma, endothelial sarcoma, lymphangiosarcoma, lymphangioendotheliosarcoma, synovial tumor, mesothelioma, Ewing's tumor, smooth muscle sarcoma, rhabdomyosarcoma, colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, cystic adenocarcinoma, medullary carcinoma, bronchial carcinoma, renal cell carcinoma, hepatocellular carcinoma, cholangiocarcinoma, choriocarcinoma, spermatogonial cell tumors, embryonal carcinomas, nephroblastomas, cervical carcinomas, testicular tumors, lung carcinomas, small-cell lung carcinomas, bladder carcinomas, epithelial carcinomas, glioma, astrocytomas, medulloblastomas, craniopharyngiomas, and ventricular tubular tumors, pineal tumors, hemangioblastomas, acoustic neuromas, oligodendrogliomas, meningiomas, melanomas, neuroblastomas, retinoblastomas, Hodgkin's disease, non-Hodgkin's lymphomas, multiple myeloma, neuroblastomas, breast cancers, rhabdomyosarcomas, primary thrombocythemia, primary macroglobulinemia, small-cell lung tumors, primary brain tumors, gastric cancers, colon cancers, malignant pancreatic islet tumors, malignant carcinoid tumors, premalignant skin lesions, testicular cancer, lymphoma, thyroid cancer, neuroblastoma, esophageal cancer, genitourinary tract cancer, malignant hypercalcemia, cervical cancer, endometrial cancer, and adrenocortical carcinoma. Preferably, the BCL2L12-related diseases suitable for treatment by the method of the present invention are ovarian cancer, melanoma, glioma, sarcoma, gastric cancer, pancreatic cancer, liver cancer, breast cancer, lung cancer, kidney cancer, adrenocortical cancer, prostate cancer, especially ovarian or liver cancer.

The present invention also provides an antisense oligonucleotide, which is 5 to 40 nucleotides in length and has at least 50% sequence identity to a reverse complement of a natural sense of a BCL2L12 polynucleotide. In one of the embodiments of the present invention, said antisense oligonucleotide is as defined as aforementioned.

The present invention also provides a pharmaceutical composition for treating or preventing a BCL2L12-associated disease in a mammal comprising the antisense oligonucleotide as defined as aforementioned, and a pharmaceutically acceptable diluent or carrier.

The present invention also provides the use of the aforementioned antisense oligonucleotides in the preparation of medicaments for treating or preventing BCL2L12-associated diseases in mammals.

The antisense oligonucleotide provided by the present invention can be used in a pharmaceutical composition by adding an effective amount of the compound to a suitable pharmaceutically acceptable diluent or carrier. For example, the antisense oligonucleotides of the invention may be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption.

Although, the antisense oligonucleotides of the present invention do not need to be administered in the context of a vector in order to modulate a target expression and/or function, embodiments of the invention relates to expression vector constructs for the expression of antisense oligonucleotides, comprising promoters, hybrid promoter gene sequences and possess a strong constitutive promoter activity, or a promoter activity which can be induced in the desired case.

The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carriers or excipients. In general, the formulations are prepared by uniformly and intimately bringing into association the active ingredients with liquid carriers or finely divided solid carriers or both, and then, if necessary, shaping the product.

### BRIEF DESCRIPTION OF THE DRAWINGS

FIG. 1 is a graph showing the expression and clinical significance of BCL2L12 gene in tumors.

FIG. 1A is the analysis diagram of BCL2L12 RNA expression in various types of tumors;

FIG. 1B is the expression analysis diagram of BCL2L12 in 374 cases of ovarian cancer samples in the TCGA database and 180 cases of normal ovarian tissues in the GTEx database;

FIG. 1C is the real-time quantitative PCR verification of BCL2L12 expression in ovarian cancer and tubal fimbria clinical samples;

FIG. 1D is a Kaplan-Meier plotter database analysis of the relationship between BCL2L12 expression and overall survival and progression-free survival.

FIG. 2 is a graph showing the expression, verification and prognosis analysis of the long transcript (BCL2L12-L) and short transcript (BCL2L12-S) of BCL2L12 in Example 2.

FIG. 2A is a schematic diagram of BCL2L12 long and short transcripts, in which the yellow area represents the BH3-like domain, the blue area represents the BH2 domain, the red area represents the abnormal coding sequence due to exon skipping, and the PTC indicates the premature termination codon.

FIG. 2B is the heat map of expression verification of BCL2L12 transcripts in ovarian cancer samples.

FIG. 2C is the analysis of the expression of BCL2L12 main transcripts in TCGA database of ovarian cancer specimens and GTEx database of normal ovarian specimens by bioinformatics analysis: the proportion of BCL2L12-L is high in ovarian cancer, and the proportion of BCL2L12-Sis low.

FIGS. 2D and E show BCL2L12 short transcripts (BCL2L12-S) are more susceptible to UPF1-core mediated mRNA degration due to the premature termination codon.

FIG. 2F shows the relationship between the expression level of exon 3 of BCL2L12 and the overall survival of ovarian cancer patients.

FIG. 3 shows the analysis diagram of the effect of antisense oligonucleotides in the junction region of BCL2L12 variable exon 3 and intron 3 on exon skipping and BCL2L12 expression.

FIG. 3A is a schematic diagram of the designed location of the two antisense oligonucleotides.

FIG. 3B is a semi-quantitative PCR verification of the regulation of the alternative splicing pattern of the two antisense oligonucleotides on the RNA of the BCL2L12 gene.

FIG. 3C shows western blotting to verify the control of two antisense oligonucleotides on BCL2L12 protein and a grayscale statistical map thereof.

FIG. 3D shows the use of ovarian cancer cell lines A2780 and HEY to verify the effect of antisense oligonucleotide ASO2 on exon skipping.

FIG. 4 shows the study of apoptosis induced by the antisense oligonucleotides provided by the present invention to regulate exon skipping of BCL2L12.

FIG. 4A is the figure of the influence of antisense oligonucleotide ASO2 on the regulation of BCL2L12 and the apoptosis level of ovarian cancer under different concentration gradients (0, 25, 50, 100, 200 nM).

FIG. 4B shows effect of antisense oligonucleotide ASO2 on the regulation of BCL2L12 and the level of apoptosis in ovarian cancer under different time (0, 24, 48, 72h).

FIG. 4C shows the effect of graph of apoptosis and inhibition of proliferation of ovarian cancer cells induced by antisense oligonucleotide ASO2 observed under light microscope; and FIG. 4D shows a graph of the proportion of apoptotic cells induced by antisense oligonucleotide ASO2 in the ovarian cancer cell lines A2780 and HEY as detected by flow cytometry.

FIG. 5 shows inhibition of ovarian cancer cell proliferation by antisense oligonucleotides provided by the present invention that regulate BCL2L12 exon skipping.

In particular, FIG. 5A shows a graph of the proportion of proliferating cells after verifying the effect of the antisense oligonucleotide ASO2 in ovarian cancer cell lines A2780, HEY, and ascites-derived ovarian cancer progenitor cells utilizing the EdU assay; and FIG. 5B shows a graph of the determination of the IC50 of the antisense oligonucleotide ASO2 on ovarian cancer cells.

FIG. 6 shows in vivo experiments using a nude mouse tumorigenic model to validate that the antisense oligonucleotide ASO2 provided by the present invention that regulates BCL2L12 exon skipping inhibits ovarian cancer growth.

FIG. 6A-FIG. 6C show graphs of subcutaneous tumorigenesis in nude mice of the HEY cell line and its volume and weight statistics, wherein after intratumoral injection of the antisense oligonucleotide ASO2, the tumor volume and weight were reduced and the ovarian cancer growth was inhibited.

FIG. 6D shows graphs of immunohistochemistry verifying the expression of the proliferation markers Ki67 and BCL2L12 in the subcutaneous tumorigenesis after the action of the antisense oligonucleotide ASO2, as well as the graph of the TUNEL assay verifying the percentage of apoptotic cells in subcutaneous tumor formation.

FIG. 7 shows inhibition of hepatocellular carcinoma cell proliferation by antisense oligonucleotides provided by the present invention that regulate BCL2L12 exon skipping.

FIG. 7A shows the regulation of BCL2L12 by different concentration gradients (25, 50, 100, 200 nM) of antisense oligonucleotide ASO2 in hepatocellular carcinoma cell line HepG2.

FIG. 7B shows western blot to verify the effect of ASO2 on BCL2L12 protein expression.

FIG. 7C shows that the decreasing level in BCL2L12 expression increased with the prolongation of ASO2 treatment.

FIG. 7D shows the inhibitory effect of ASO2 on cell proliferation detected by MTT assay.

FIG. 7E shows the proliferation inhibition and killing effect of ASO2 on hepatocellular carcinoma cells observed under light microscopy.

### DETAILED DESCRIPTION OF EMBODIMENTS

The present invention will be further described below, but these examples do not limit the invention in any respect.

### Example 1 Expression of BCL2L12 and its Correlation with Clinical Prognosis

Based on the gene expression profile data in the TCGA and GTEx databases, the expression of BCL2L12 in each tumor was analyzed.

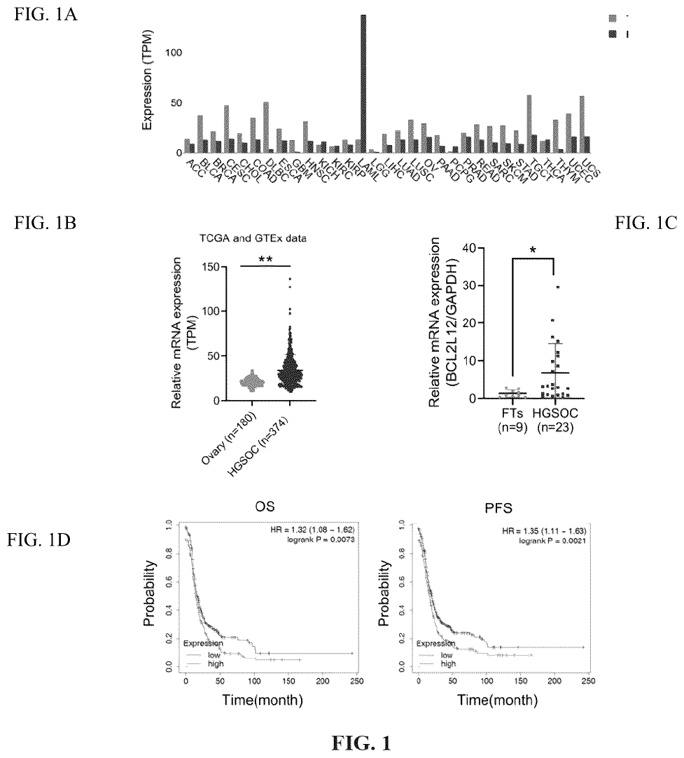

TPM (Transcripts Per Million) was used to measure the level of RNA expression, and the average expression of all samples was calculated to detect the expression of BCL2L12 in 30 kinds of tumors. The results are shown in FIG. 1A. The results showed that BCL2L12 was highly expressed in various tumors.

The prognosis of tumors was further analyzed. Using the Kaplan-Meier Plotter (http://kmplot.com) and GEPIA2 (http://gepia2.cancer-pku.cn) data, the expression of BCL2L12 was divided into high and low groups, and the optimal cut-off value was selected. The relationship between the expression of BCL2L12 and the overall survival rate of patients was analyzed. The results showed that high expression of BCL2L12 was associated with poor prognosis (hazard ratio>1). Among them, skin melanoma, brain low-grade glioma, sarcoma, gastric cancer, pancreatic cancer, liver cancer, ovarian serous cystadenocarcinoma, breast invasive carcinoma, lung adenocarcinoma, renal papillary cell carcinoma, adrenal cortical carcinoma, and prostate adenocarcinoma showed a significant correlation between high expression of BCL2L12 and poor prognosis (p-value<0.05).

The expression of BCL2L12 in ovarian cancer was analyzed in detail. Information from 180 GTEx-ovary normal ovarian tissues and 374 TCGA-OV ovarian cancer tissues in the database was used. The results are shown in FIG. 1B, it can be seen that BCL2L12 is highly expressed in ovarian cancer.

The research group of the inventors of the present application collected fimbria and ovarian cancer samples from Qilu Hospital of Shandong University. Ovarian cancer samples were obtained from patients with primary ovarian cancer who had not undergone any surgery or chemotherapy. In addition, normal fimbria samples were obtained from patients who underwent total hysterectomy and bilateral salpingo-oophorectomy due to uterine disease or pathological changes of benign tumor adnexa. This study was approved by the Ethics Committee of Shandong University (SDULCLL2019-1-09), and all patients provided written informed consent.

The expression level of BCL2L12 gene was further verified by real-time quantitative PCR. The primers for amplifying the BCL2L12 gene are as follows:

BCL2L12-F:
AAGACACGCTGAGGGTCCTA
BCL2L12-R:
GGTGGAGTTGGAACAGGAGA

The results are shown in FIG. 1C.

Further, based on the Kaplan-Meier plotter database, the patients' BCL2L12 gene expression and clinical information were correlated, and the correlation between the two was analyzed. The results are shown in FIG. 1D. From the analysis results of Overall survival (OS) and Progression-Free-Survival (PFS), it can be seen that high expression of BCL2L12 is closely related to poor prognosis (p<0.01).

### Example 2 BCL2L12 Transcript Difference and its Expression

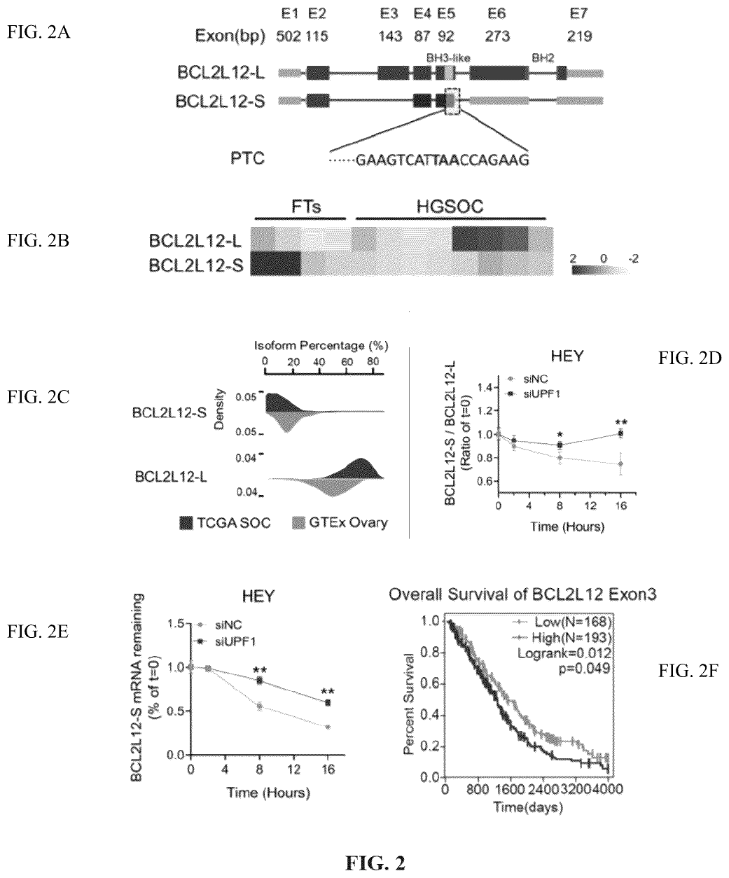

FIG. 2A is a schematic diagram of the gene sequence and structure of BCL2L12 and its two main transcripts: long transcript (BCL2L12-L) and short transcript (BCL2L12-S), which shows the exons, introns and regions of BCL2L12 functional domain.

Using 8 cases of ovarian cancer samples and 4 cases of normal fimbria samples collected by the inventor's research group of the present application, real-time quantitative PCR was performed on the expression of BCL2L12-L and BCL2L12-S, and the relative expression was compared using the ΔCT method, and the heatmap package was used to reveal their relative expression ratios (as shown in FIG. 2B).

BCL2L 12-L and BCL2L12-S specific primers are:
BCL2L12-L-F:
CCCAAGAAGAGCCAACAGAC
BCL2L12-L-R:
CAGGGAGCAGGGAAGACATC
BCL2L12-S-F:
TCCACCTAGGCCCAGCTAC
BCL2L12-S-R:
CGGAGATTTCAGCTGCTCTT

Based on the UCSC Xena database, further analysis of the expression profiles of ovarian cancer samples in the TCGA database and normal ovarian samples in the GTEx database showed that the proportion of BCL2L12-L in ovarian cancer was significantly higher than that of the short transcript BCL2L12-S. BCL2L12-S has a premature termination codon (PTC), meeting the definition of a nonsense-mediated mRNA degradation (NMD) transcript.

UPF1 is a key protein in the complex that degrades NMD transcripts. The following siRNA sequence was synthesized from Genepharma LTD.:

UPF1 #1:
5′-GAUGCAGUUCCGCUCCAUU-3′

UPF1 was knocked down by transient transfection of siUPF1, and after 48 h, actinomycin D was added at a final concentration of 5 μg/ml to terminate transcription, so as to explore the degradation rate of BCL2L12-L and BCL2L12-S. Take the beginning of adding actinomycin D as t=0, and compare with it. It was found that BCL2L12-S with skipped exon 3 was degraded faster, and its degradation was slowed down after knockdown of UFP1 (FIGS. 2D and E).

Using the transient transfection technique, siUPF1 was transfected into the HEY ovarian cancer cell line, and actinomycin D was added to stop the transcription, so as to verify the effect of knocking down UPF1 on RNA stability. The transient transfection steps mainly include:
1. Cell plating: Take the cells that have been passaged twice, and the cells in good condition are plated and transfected. Trypsinization-medium resuspended cells-cell count, seeding cell density. When transfecting the plasmid, the cell density was controlled at 60%-80%.
2. Prepare the transfection complex: DNA transfection method: Mix 5 μl dissolved siRNA (125 μl DEPC water dissolved 2OD siRNA) with 200 μl jetPRIME Buffer and add it to a 1.5 ml EP tube, vortex for 10 seconds, add 4 μl jetPRIME, vortex again for 10 seconds and centrifuge. Incubate at room temperature for 10 min.
3. Add the above-mentioned transfection mixture to the 6-well plate that has been inoculated with cells, add 200 μl of the above-mentioned mixture to 2 ml of culture medium per well, mix well and put it into a cell culture incubator.
4. After completing the verification of UPF1 knockdown efficiency, 48 hours after transfection, the transcription inhibitor actinomycin D at the same concentration (5 μg/ml) was added sequentially in chronological order, and RNA was extracted after incubation, and real-time quantitative PCR was performed.

In addition, the relationship between the expression level of exon 3 of BCL2L12 and the overall survival of patients was analyzed. Based on the UCSC Xena database, the expression levels of BCL2L12 exons in the TCGA database of ovarian cancer samples were obtained, and the clinical prognosis information of ovarian cancer patients in the TCGA database was obtained at the same time. The correlation analysis between the two was performed through the program package in R language Survival and survminer, and the result graph visualization is realized based on R Studio. The results showed that the expression level of exon 3 was significantly correlated with poor prognosis (p-value<0.05) (FIG. 2F), while the expression level of other exons had no statistically significant relationship with prognosis. Therefore, the quantitative analysis of exon 3 of BCL2L12 can be used to judge the prognosis of ovarian cancer patients.

### Example 3 Antisense Oligonucleotide Targeting BCL2L12 Exon 3 and its Effect

The inventors of the present application found that the difference in the core transcripts of BCL2L12 lies in exon 3, and found a classic GU splice site surrounding the junction of exon 3 and intron 3 of BCL2L12. The inventors designed and prepared antisense oligonucleotides located at the junction region of exon 3 and intron 3 of BCL2L12:

(SEQ ID NO: 2)
GAAATCTCTTACCAGGCTCTAAAC
(SEQ ID NO: 3)
CATAGATGATCATGGAAATCTC

By complementary base pairing, antisense oligonucleotides hinders the splicing of immature precursor RNA of downstream target genes by splicing elements, so as to interfere with the splicing of BCL2L12 and cause changes in the splicing pattern of exon 3.

Entrust Beijing Qingke Biotechnology Co., Ltd. to synthesize the following antisense oligonucleotides:

BCL2L12-ASO-2:
G*A*A*A*T*C*T*C*T*TACCAGG*C*T*C*T*A*A*A*C
BCL2L12-ASO-3:
C*A*T*A*G*A*T*GATCATG*G*A*A*A*T*C*T*C
BCL2L12-NC (negative control):
C*C*T*C*T*T*A*C*C*TCAGTTA*C*A*A*T*T*T*A*T*A.

*represents phosphorothioate modification.

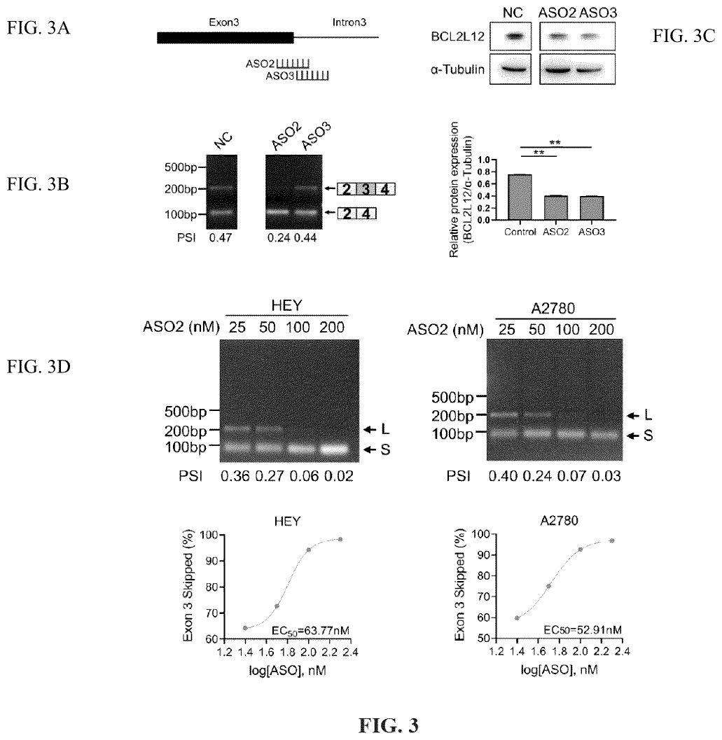

FIG. 3A shows the antisense oligonucleotides BCL2L12-ASO-2 and BCL2L12-ASO-3 binding sites. As shown in the figure, BCL2L12-ASO-3 is reverse complementary to the 5′ end sequence of intron 3 of BCL2L12 immature pre-mRNA. BCL2L12-ASO-2 is reverse complementary to the junction site of exon 3 and intron 3 and the flanking sequences.

150 nM of the aforementioned antisense oligonucleotides were transiently transferred into the ovarian cancer cell line A2780 using jetPRIME, and the cellular RNA was collected after 48 hours, and the cellular protein was collected after 72 hours. The changes in RNA and protein levels were verified by semi-quantitative PCR and Western blotting, respectively. Total cellular RNA was extracted using a Total RNA Extraction Kit (Chengdu Fuji Biotechnology Co., Ltd.), and reverse-transcribed using HiScript II Q Select RT SuperMix for qPCR (Nanjing Nuoweizan Biotechnology Company, R232-01), at 37° C. 15 minutes, 85° C. for 5 seconds, the reversed cDNA was amplified using semi-quantitative PCR primers (see below) and 2×Taq Plus Master Mix (Dye Plus) (Nanjing Novizym Biotechnology Co., Ltd., P212-01), and the product was subjected to 1.5% agarose gel electrophoresis, 110V for 30 minutes and take pictures.

BCL2L12-E2-F
5′-TCTCCTGTTCCAACTCCACC-3′
BCL2L12-E4-R
5′-TTTCAGCTGCTCTTGGACCA-3′.

The results are shown in FIG. 3B and FIG. 3C. Transient transfection of ovarian cancer cell lines with ASO2 and ASO3 produced more nonsense-mediated degradation (NMD)-sensitive BCL2L12-S and decreased BCL2L12 protein levels. In the semi-quantitative PCR, the splicing pattern of the RNA level of BCL2L12 was quantitatively calculated using the Percent Spliced In (PSI), and the grayscale analysis of the image after agarose electrophoresis was performed using ImageJ:

Percent Spliced In (PSI)=Grayscale of Exon Inclusion Band/(Grayscale of Exon Inclusion Band+Grayscale of Exon Skipping Band)

The inventors further tested the effects of antisense oligonucleotide ASO2 with different concentration gradients on ovarian cancer cell lines A2780 and HEY. The median effect concentration (EC50) of antisense oligonucleotide ASO2 on exon skipping was calculated by counting the percentage of exon skipping. The results were shown in FIG. 3D. Antisense oligonucleotide ASO2 could promote the skipping of exon 3 of BCL2L12 in a concentration-dependent manner. The median effect concentration (EC50) of antisense oligonucleotide ASO2 on exon skipping was 63.77 nM and 52.91 nM in HEY and A2780 cell lines, respectively.

The percentage of exon skipping (%)=Grayscale of Exon Skipping Band/Grayscale of Exon Inclusion Band+Grayscale of Exon Skipping Band)

### Example 4 Antisense Oligonucleotide Induces Ovarian Cancer Apoptosis and Inhibits Proliferation Thereof

Based on that ASO2 can regulate the characteristics of BCL2L12 alternative splicing and down-regulation of BCL2L12 protein, further study its induction of tumor cell apoptosis and anti-tumor effect.

Set the final concentration gradient of 0, 25, 50, 100, 200 nM, and the time gradient of 0, 24, 48, 72h, and use Western blotting to verify the protein expression level of BCL2L12 and the key apoptosis marker cleaved-caspase3.

The main experimental steps of Western blotting are:
1. Loading sample preparation: Prepare 30 μg of protein sample that is fully lysed and centrifuged with total cell protein lysate, dilute the sample with 5×SDS, denature by heating at 95° C. for 10 minutes, and cool on ice.
2. Polyacrylamide gel (SDS-PAGE) electrophoresis: the loading volume of the protein marker is 2.5 μl, and the loading volume of the other wells is controlled within 25 μl. Electrophoresis was performed with a voltage of 60-80V.
3. Membrane transfer (wet transfer method): Cut out a PVDF membrane that is similar in shape and size to the gel, activate it with methanol, and soak it in the transfer buffer for 10-15 minutes to balance. The sandwich arrangement method is adopted: the PVDF membrane is placed on the SDS-PAGE gel, and the sponge is attached to the upper and lower sides. Place it tightly, and put the fixed plate into the transfer film tank. Place the film transfer tank in an ice-water bath, and transfer the film at 200 mA for 60-90 minutes.
4. Blocking: block with 5% skimmed milk in TBST solution for 1 hour on a shaker.
5. Incubate with the primary antibody and add BCL2L12 (Abways, CY9733) and Cleaved-caspase3 (Cell Signaling Technology, #9661) primary antibody diluted with Western primary antibody diluent (Shanghai Beyontian Biotechnology Co., Ltd.) at a ratio of 1:1000. The PVDF membrane with the protein sample was snapped back onto the primary antibody.
6. Secondary antibody incubation: wash the membrane three times in TBST solution with 1% Tween 20, each time for 10 minutes, and dilute the secondary antibody (Jackson, 115-025-003) with blocking solution at 1:5000. Incubate on a shaker at room temperature for 1 hour. Wash three times in TBST, 10 minutes each time.
7. Chemiluminescence: Chromogenic solution A and B were freshly prepared at a ratio of 1:1, and an appropriate chromogenic solution was added and evenly dropped onto the membrane, and imaged with an ECL chemiluminescent system.

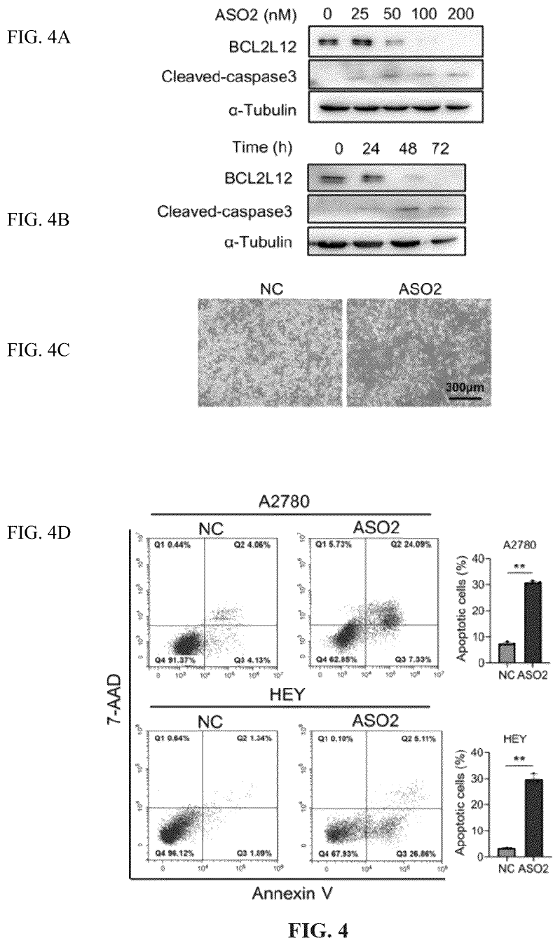

The results were shown in FIG. 4A. With the increase of ASO2, the expression of BCL2L12 protein decreased, and at the same time, all concentration gradients could produce cleaved-caspase3, thereby causing cell apoptosis. In addition, as shown in FIG. 4B, ASO2 began to work 24 hours after the transient transfection, and apoptotic cells were generated. As the action time was extended to 72 hours, the knockdown efficiency of BCL2L12 protein level was the most obvious, and the number of apoptotic cells increased gradually.

Cell viability, proliferation and apoptosis were observed under a microscope. The results are shown in FIG. 4C. As an example, ovarian cancer cells treated 48 hours of treatment with 150 nM antisense oligonucleotide ASO2 showed obvious apoptosis.

Use the Annexin V-PE/7-AAD Apoptosis Staining Kit (Nanjing Novizan Biotechnology Co., Ltd.) to quantitatively determine the percentage of apoptosis. The specific assay steps are as follows:
1. Collect cells: collect 5×10⁵ adherent cells, digest the cells with EDTA-free trypsin, collect the cells after termination of digestion, centrifuge at 1000 rpm, 4° C. for 5 minutes, and discard the supernatant.
2. Wash the cells: Wash the cells twice with pre-cooled PBS, centrifuge at 1000 rpm and 4° C. for 5 minutes each time, and discard the supernatant.
3. Resuspend the cells: add 100 μl 1×Binding Buffer, and blow gently to obtain a single cell suspension.
4. Cell staining: add 5 μl Annexin V-PE and 5 μl 7-AAD Staining Solution and blow evenly; incubate at room temperature (20-25° C.) for 10 minutes; add 400 μl 1×Binding Buffer and mix well.
5. The samples were detected and analyzed by flow cytometry within 1 hour after staining: normal cells were double negative (Annexin V-PE/7-AAD); early apoptotic cells were Annexin V-PE single positive (Annexin V-PE/7-AAD); late apoptotic cells were double positive for Annexin V-PE and 7-AAD (Annexin V-PE/7-AAD).

The experimental results are shown in FIG. 4D. It was found that the antisense oligonucleotide ASO2 could induce an increase in the ratio of early apoptosis and late apoptosis, which was consistent with the change of protein expression observed under the microscope.

In addition, in different ovarian cancer cells or cell lines, the EdU cell proliferation detection kit (Guangzhou Ruibo Biotechnology Co., Ltd.) was used to verify the cell viability of the cells by the MTT method after treated with ASO2 and calculate the IC50.

Cells or cell lines used for experiments include A2780, HEY, and ascites-derived ovarian cancer primary cells. Among them, the A2780 and HEY ovarian cancer cell lines were donated by the research group of Professor Wei Jianjun of Northwestern University.

Wherein, the preparation method of ascites-derived ovarian cancer primary cells is as follows:

Ascites was obtained from the Department of Obstetrics and Gynecology, Qilu Hospital of Shandong University, and the freshly separated liquid was collected in a sterile vacuum container by the method of culturing primary ovarian cancer cells described in previous studies (Shepherd et al., 2006; Theriault et al., 2013). Afterwards, 25 ml of ascitic fluid was mixed with an equal amount of MCDB/M199 medium containing 10% fetal bovine serum in a T-75 cell culture flask. Cells were incubated for 3-4 days before the first change of complete medium. Then the medium was changed every 2-3 days until the primary cells in the cell culture flask were confluent, and the cells were passaged at a dilution ratio of 1:2, and the experiments were carried out using passage 2-6 cells. Collect the same batch of ovarian cancer primary cells, freeze them in 70% v/v MCDB/M199 medium, 20% v/v fetal bovine serum and 10% dimethyl sulfoxide, use the gradient cooling method, and finally store them in liquid in nitrogen.

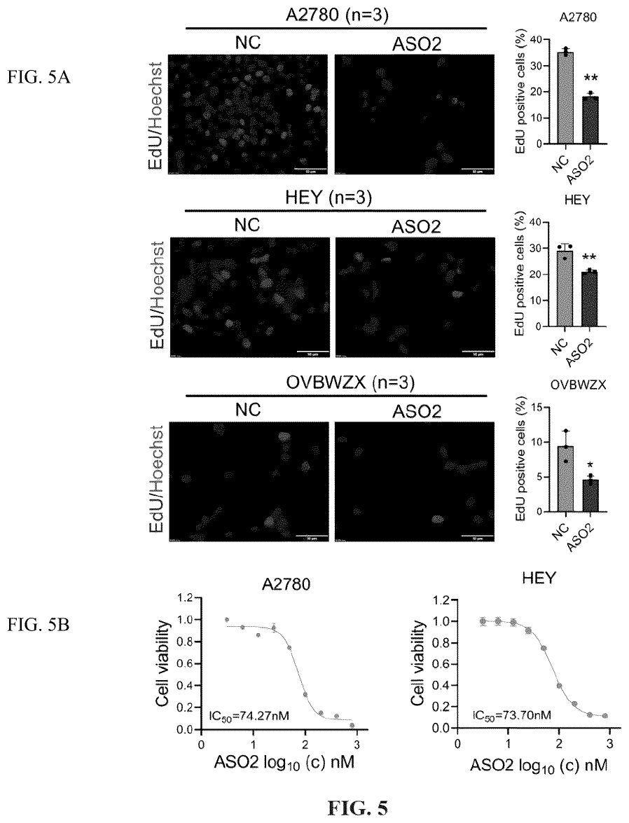

The result is shown in FIG. 5. FIG. 5A is a diagram of the ratio of proliferating cells after the antisense oligonucleotide ASO2 is added, verified by EdU experiment in the ovarian cancer cell lines A2780, HEY and ascites-derived ovarian cancer primary cells. FIG. 5B is a graph showing the determination of the median inhibitory concentration IC50 of the antisense oligonucleotide ASO2 on ovarian cancer cells.

The results showed that after the antisense oligonucleotide ASO2 was transiently transfected into ovarian cancer cell lines A2780, HEY, and ascites-derived ovarian cancer primary cells, the proportion of EdU-positive cells labeled with the red dye Apollo567 decreased, showing ASO2 significantly inhibited the proliferation of tumor cells rate. The inhibitory effect of antisense oligonucleotide ASO2 on ovarian cancer cell lines A2780 and HEY was determined, and the median inhibitory concentrations (IC50) were 73.70 nM and 74.27 nM, respectively.

### Example 5 In Vivo Experiments Verify that Antisense Oligonucleotides Inhibit the Growth of Ovarian Cancer Cells

BALB/c nude female mice aged 4-8 weeks were subjected to subcutaneously injection of ovarian cancer HEY cells 1×10⁶ cells/place in the armpits bilaterally. When the subcutaneous tumor diameter was about 3-5 mm, 7 nude mice with the same size on both sides were selected. Intratumoral injection of antisense oligonucleotide ASO2 was performed.

Each nude mouse was injected with 5 nmol of antisense oligonucleotide, diluted with 25 μl Opti-MEM and mixed with 3 μl lipo2000, injected once every three days and observed and recorded the tumor size. After 2-3 weeks, the nude mice were anesthetized and sacrificed, and the subcutaneous tissue was taken. Tumor tissues were soaked in 10% formalin solution, embedded in paraffin, and then sectioned for HE staining and immunohistochemical staining to determine the tumor proliferation rate, apoptosis level, and BCL2L12 expression.

HE staining steps are as follows:

Dry the slices in an oven at 60 degrees for 30 minutes and then dewax, xylene I for 15 minutes, xylene II for 15 minutes, 100% alcohol I for 5 minutes, 100% alcohol II for 5 minutes, 95% alcohol for 5 minutes, 80% alcohol for 5 minutes, and 75% alcohol for 5 minutes Minutes, rinse in tap water 2-3 times.

Stain with hematoxylin for 10 minutes and gently rinse the slides with a small stream of water. Hydrochloric acid alcohol for 3 seconds, ammonia water for 5-10 seconds, and tap water for 2-3 times. Stain with eosin for 20 minutes and rinse with tap water 2-3 times.

Dehydration: 75% alcohol for 10 s, 80% alcohol for 10 s, 95% alcohol for 10 s, 100% alcohol for 10 s, 100% alcohol for 10 s, xylene I for 3 minutes, xylene II for 3 minutes.

### 2. Immunohistochemical Staining

The dewaxing step is the same as above.

Antigen restoration: add restoration solution (pH=8.0 EDTA), put it in a washing box, heat it in a microwave oven on high heat for 10 minutes until it boils, immediately put the dewaxed slices in, heat it in a microwave oven for 15 minutes, and then let it cool down to room temperature naturally.

Immunohistochemical kit (Beijing Zhongshan Jinqiao Biotechnology Co., Ltd.): incubate with 3% hydrogen peroxide in a wet box at 37 degrees for 15-20 minutes, wash with PBS shaker 3 times for 3 minutes; serum blocking: drop reagent A (blue liquid)) and incubate at 37° C. for 25-30 min; add primary antibody: room temperature for 2 hours, add reagent B: incubate at 37° C. for 15-20 min, wash with PBS shaker for 3 times*3 min; add reagent B: incubate at 37° C. for 10-15 min, PBS shaker Wash 3 times*3 min.

Add the chromogenic solution DAB and incubate for 5-10 minutes, observe under the microscope, stain with hematoxylin, soak in hydrochloric acid alcohol for 3 seconds, and in ammonia water for 7-10 seconds; perform dehydration (the steps are the same as above).

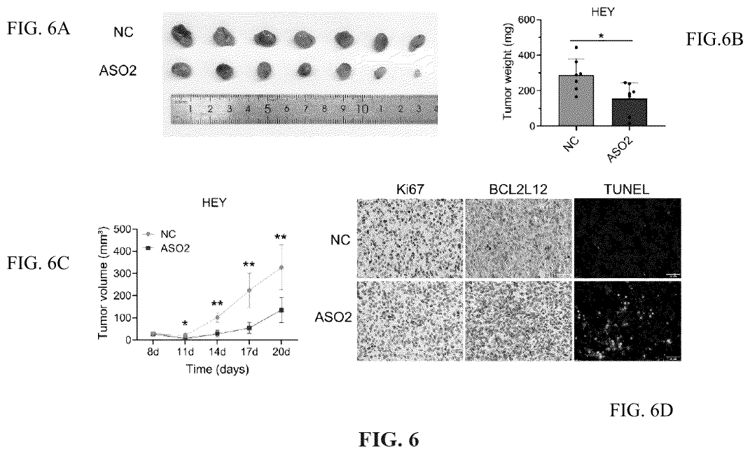

The result is shown in FIG. 6. FIG. 6A-C are pictures of subcutaneous tumor formation in nude mice, and the volume and weight of the tumors were counted after intratumoral injection of antisense oligonucleotide ASO2 and the corresponding control. The results showed that the antisense oligonucleotide ASO2 had an inhibitory effect on tumor growth. FIG. 6D shows the results of immunohistochemistry and TUNEL staining on tumor sections. The results showed that as the expression of BCL2L12 decreased, the expression of cell proliferation marker Ki67 decreased, and the level of apoptosis gradually increased.

These experiments proved that antisense oligonucleotide ASO2 can inhibit the growth of ovarian cancer cells in vivo.

### Example 6 Antisense Oligonucleotide Induces Ovarian Cancer Apoptosis and Inhibits its Proliferation

Based on that ASO2 can regulate the characteristics of BCL2L12 alternative splicing and down-regulation of BCL2L12 protein, further study its induction of liver cancer cell apoptosis and anti-tumor effect.

Use 25 nM, 50 nM, 100 nM, 200 nM ASO2 to transfect liver cancer cell line HepG2 with lipo2000, extract RNA after 48 hours, perform semi-quantitative PCR after reverse transcription, and use primers on both sides of exon 3 of BCL2L12 to detect its alternative splicing model.

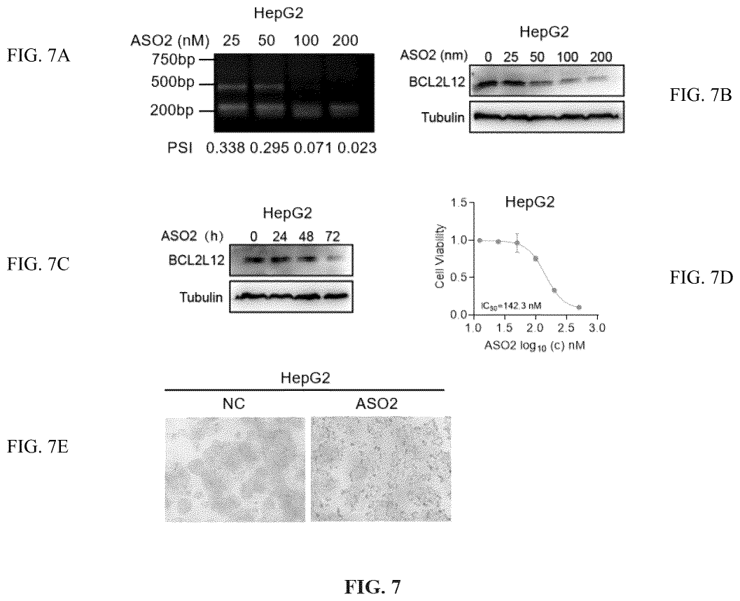

The results are shown in FIG. 7A. As the concentration of ASO2 increased, the expression of the long transcript BCL2L12-L exon 3 inclusion gradually decreased, and the PSI value gradually decreased.

At the protein level, Western blotting was used to verify the effect of ASO2 on the expression of BCL2L12 protein. The results showed that the corresponding protein expression gradually decreased with the increase of ASO2 concentration (FIG. 7B), and with the prolongation of ASO2 treatment time, the reduction range of BCL2L12 expression increased (FIG. 7C).

The inhibitory effect of ASO2 on cell proliferation was detected by MTT assay. The IC50 of ASO2 on HepG2 cells was determined to be 142.3 nM (FIG. 7D), and the proliferation inhibition and killing effect of ASO2 on liver cancer cells could also be observed under the light microscope (FIG. 7E).

The practice of the present invention will employ, unless otherwise indicated, conventional techniques of biotechnology, organic chemistry, inorganic chemistry, etc., and it will be apparent that the invention can be carried out otherwise than as specifically described in the foregoing specification and examples. Other aspects and modifications within the scope of the invention will be apparent to those skilled in the art to which the invention pertains. Many modifications and variations are possible based on the teachings of the present invention and are therefore within the scope of the present invention. All patents, patent applications, and scientific papers mentioned herein are hereby incorporated by reference.

1.-14. (canceled)

15. A method of modulating a function of and/or the expression of BCL2L12 polynucleotides in mammalian cells or tissues in vivo or in vitro, comprising: contacting said mammalian cells or tissues with an antisense oligonucleotide 5 to 40 nucleotides in length, wherein said antisense oligonucleotide has at least 50% sequence identity to a reverse complement of a natural sense of a polynucleotide fragment at the junction of exon 3 and intron 3 of a BCL2L12 polynucleotide, thereby modulating a function of and/or the expression of the BCL2L12 polynucleotides.

16. The method of claim 15, wherein said antisense oligonucleotide has at least 50% sequence identity to a reverse complement of a polynucleotide comprising 5 to 40 consecutive nucleotides within the nucleotides 1567 to 1626 of a BCL2L12 polynucleotide of SEQ ID NO: 1.

17. The method of claim 16, wherein said antisense oligonucleotide has at least 50% sequence identity to a reverse complement of a natural sense of the following polynucleotide fragments at the junction of exon 3 and intron 3 of the BCL2L12 polynucleotide: a fragment of about 5-40 nucleotides at the end of intron 3 connecting with exon 3, a fragment of about 5-40 nucleotides at the end of exon 3 connecting with intron 3, or a fragment of about 5-40 nucleotides at the junction of exon 3 and intron 3.

18. The method of claim 17, wherein said antisense oligonucleotide includes a reverse complement of a natural sense of the polynucleotide fragment of about 5 nucleotides at the end of intron 3 connecting with exon 3 of the BCL2L12 polynucleotide.

19. The method of claim 18, wherein said antisense oligonucleotide includes a reverse complement of the nucleotides 1597 to 1601 of a BCL2L12 polynucleotide of SEQ ID NO: 1.

20. The method of claim 15, wherein said antisense oligonucleotide comprises one or more modifications selected from: at least one modified sugar moiety, at least one modified internucleoside linkage, at least one modified nucleotide, and combinations thereof.

21. The method of claim 20, wherein the one or more modifications comprise at least one modified internucleoside linkage selected from: a phosphorothioate, 2′-Omethoxyethyl (MOE), 2′-fluoro, alkylphosphonate, phosphorodithioate, alkylphosphonothioate, phosphoramidate, carbamate, carbonate, phosphate triester, acetamidate, carboxymethyl ester, and combinations thereof.

22. The method of claim 15, wherein said antisense oligonucleotide has a nucleotide sequence as shown in SEQ ID NO:2 or SEQ ID NO:3.

23. The method of claim 22, wherein said oligonucleotide is:
G*A*A*A*T*C*T*C*T*TACCAGG*C*T*C*T*A*A*A*C;
or
C*A*T*A*G*A*T*GATCATG*G*A*A*A*T*C*T*C
(* stands for phosphorothioate modification).

24. The method of claim 15, wherein said antisense oligonucleotide binds to the natural sense BCL2L12 polynucleotide in a cell or tissue of said mammal and modulates the BCL2L12-L transcript and the BCL2L12-S transcript.

25. The method of claim 24, wherein said antisense oligonucleotide reduces the expression of BCL2L12.

26. The method of claim 15, wherein said method is for treating or preventing a BCL2L12-associated disease in a mammal.

27. The method of claim 26, wherein said BCL2L12-associated disease is a cancer selected from a group consisting of: fibrosarcoma, mucinous sarcoma, liposarcoma, chondrosarcoma, osteogenic sarcoma, chordoma, hemangiosarcoma, endothelial sarcoma, lymphangiosarcoma, lymphangioendotheliosarcoma, synovial tumor, mesothelioma, Ewing's tumor, smooth muscle sarcoma, rhabdomyosarcoma, colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, cystic adenocarcinoma, medullary carcinoma, bronchial carcinoma, renal cell carcinoma, hepatocellular carcinoma, cholangiocarcinoma, choriocarcinoma, spermatogonial cell tumors, embryonal carcinomas, nephroblastomas, cervical carcinomas, testicular tumors, lung carcinomas, small-cell lung carcinomas, bladder carcinomas, epithelial carcinomas, glioma, astrocytomas, medulloblastomas, craniopharyngiomas, and ventricular tubular tumors, pineal tumors, hemangioblastomas, acoustic neuromas, oligodendrogliomas, meningiomas, melanomas, neuroblastomas, retinoblastomas, Hodgkin's disease, non-Hodgkin's lymphomas, multiple myeloma, neuroblastomas, breast cancers, rhabdomyosarcomas, primary thrombocythemia, primary macroglobulinemia, small-cell lung tumors, primary brain tumors, gastric cancers, colon cancers, malignant pancreatic islet tumors, malignant carcinoid tumors, premalignant skin lesions, testicular cancer, lymphoma, thyroid cancer, neuroblastoma, esophageal cancer, genitourinary tract cancer, malignant hypercalcemia, cervical cancer, endometrial cancer, and adrenocortical carcinoma.

28. The method of claim 27, wherein the cancer is ovarian or liver cancer.

29. An antisense oligonucleotide, which is 5 to 40 nucleotides in length and has at least 50% sequence identity to a reverse complement of a natural sense of a polynucleotide fragment at the junction of exon 3 and intron 3 of a BCL2L12 polynucleotide.

30. The antisense oligonucleotide of claim 29, wherein said antisense oligonucleotide has at least 50% sequence identity to a reverse complement of a polynucleotide comprising 5 to 40 consecutive nucleotides within the nucleotides 1567 to 1626 of a BCL2L12 polynucleotide of SEQ ID NO: 1.

31. The antisense oligonucleotide of claim 29, wherein said antisense oligonucleotide has at least 50% sequence identity to a reverse complement of a natural sense of the following polynucleotide fragments at the junction of exon 3 and intron 3 of the BCL2L12 polynucleotide: a fragment of about 5-40 nucleotides at the end of intron 3 connecting with exon 3, a fragment of about 5-40 nucleotides at the end of exon 3 connecting with intron 3, or a fragment of about 5-40 nucleotides at the junction of exon 3 and intron 3.

32. The antisense oligonucleotide of claim 29, wherein said antisense oligonucleotide includes a reverse complement of a natural sense of the polynucleotide fragment of about 5 nucleotides at the end of intron 3 connecting with exon 3 of the BCL2L12 polynucleotide.

33. The antisense oligonucleotide of claim 29, wherein said antisense oligonucleotide comprises at least one modified internucleoside linkage selected from: a phosphorothioate, 2′-Omethoxyethyl (MOE), 2′-fluoro, alkylphosphonate, phosphorodithioate, alkylphosphonothioate, phosphoramidate, carbamate, carbonate, phosphate triester, acetamidate, carboxymethyl ester, and combinations thereof.

34. The antisense oligonucleotide of claim 29, wherein said antisense oligonucleotide has a nucleotide sequence as shown in SEQ ID NO:2 or SEQ ID NO:3.
